(19) United States
(12) Patent Application Publication
(10) Pub. No.: US 2006/0045281 A1
(43) Pub. Date: Mar. 2, 2006

(54) Parameter adjustment in audio devices

(75) Inventors: Jose E. Korneluk, Boynton Beach, FL (US); Marc A. Boillot, Plantation, FL (US); Jose C. Lacal, Boynton Beach, FL (US); John G. Harris, Gainesville, FL (US)

Correspondence Address: POSZ LAW GROUP, PLC, 12040 SOUTH LAKES DRIVE, SUITE 101, RESTON, VA, 20191, US

(73) Assignee: MOTOROLA, INC.

(21) Appl. No.: 10/928,670

(22) Filed: Aug. 27, 2004

(51) Int. Cl. H04R 29/00; A61B 5/00

(52) U.S. Cl. 381/60; 600/559

(57) ABSTRACT

A method (400, 600) and apparatus (500, 800) allow adjusting parameters associated with an audio signal output from a device. Tones are output according to a first interactive test profile presented on a user interface during a first test period. A first interaction forms a first adjustment profile with adjustment levels. The audio signal is output with parameters adjusted according to the first adjustment profile. The audio signal is output from the device in accordance with the first adjustment profile and a second interactive test profile. An interaction with the user interface forms a second adjustment profile having second adjustment levels and the audio signal is then adjusted in accordance with the second adjustment levels. The second interactive test profile includes a speech sample and an intelligibility parameter including a spectral tilt for the device and a formant sharpening profile.

### FIELD OF THE INVENTION

The present invention relates in general to audio adjustment and equalization, and more specifically to a method and apparatus for self-adjusting audio levels and providing equalization for communications-related devices and other audio devices.

### BACKGROUND OF THE INVENTION

With the widespread proliferation of portable communications devices such as, for example, cellular handsets, and portable audio players such as, for example, portable Compact Disc (CD) players or MPEG 1 (Motion Picture Experts Group) Layer 3 (MP3) player, portable computers, Portable Digital Assistants (PDAs) and the like, the quality of audio has become an important factor in maximizing the usability, fidelity, and enjoyment of such devices. For devices focusing on speech such as communication units, additional issues arise although many fidelity-related issues are common to speech and music.

Individuals listen to speech in unique ways, paying attention to certain voicing or accent characteristics and interpreting speech information accordingly. Some listeners may pay more attention to certain vowel sounds or vocalization styles and thus a particular speaking style or accent establishes an expectation of what will follow. By properly identifying attributes of particular interest to a listener, a better listening experience can be provided by emphasizing those attributes. Attaining a high level of audio quality for users of all kinds, including hearing impaired users is of increasing importance.

Problems arise in connection with controlling audio through the use of, for example, conventional volume and/or tone control, particularly for devices with a narrow audio band such as communication units. A typical communication unit such as a wireless handset for example, has a band limited frequency response between around 150 Hz to around 3600 Hz. Since listeners have different hearing capabilities, preferences, or the like, and since hearing impaired listeners may have hearing capabilities with very specific impairments at certain frequencies, conventional systems rarely provide adequate audio fidelity for users having hearing capabilities falling outside normal levels, or for users having specific impairments. Still further, standards and regulations require levels to fall within certain boundaries further increasing the challenge of providing adequate fidelity to those whose hearing capabilities are not within ranges typically considered “normal”.

Some hearing deficient or hearing impaired users are well aware of their deficiencies and either purchase expensive hearing aids specifically tailored to boost specific frequencies associated with their impairment or attempt to listen unassisted, often losing specific audio information such as high frequency components of the audio signal. Such frequency specific audio information loss can degrade the overall quality or intelligibility of the listening experience of, for example, a musical segment, or can result in the misinterpretation of certain sounds occurring regularly in speech such as consonants with high frequency components such as the sounds associated with the consonants “F”, “T”, “P”, “S”, and the like. Problems associated with, for example, a hearing impaired listener attempting to engage in unassisted listening are further exacerbated in devices with narrow audio bands as described above.

Therefore, to address the above described problems and other problems, what is needed is a method and apparatus for addressing issues associated with poor audio level and equalization control in devices such as handsets, including wireless handsets wired handsets, and the like, and other audio devices such as MP3 players, portable computers portable CD players, PDAs, and the like, particularly where control may concern hearing deficiencies, impairments, preferences and the like.

### BRIEF DESCRIPTION OF THE DRAWINGS

The accompanying figures, where like reference numerals refer to identical or functionally similar elements and which together with the detailed description below are incorporated in and form part of the specification, serve to further illustrate a preferred embodiment and to explain various principles and advantages in accordance with the present invention.

FIG. 1 is a diagram illustrating a simplified and representative exemplary hearing profile including frequency band and level for left and right hearing for a listener and a region associated with normal hearing levels;

FIG. 2 is a diagram further illustrating a simplified and representative exemplary hearing profile including frequency band and level for several left and right hearing profiles for listeners with different hearing impediments including low frequency hearing loss, high frequency hearing loss, and total hearing loss, and a region of normal hearing levels;

FIG. 3 is a diagram illustrating a simplified and representative exemplary hearing profile including frequency band and level for left and right hearing for a listener corrected for in accordance with various exemplary embodiments;

FIG. 4 is a flow chart illustrating an exemplary audiogram or loudness test procedure in accordance with various exemplary and alternative exemplary embodiments;

FIG. 5 is a block diagram illustrating exemplary components in a speech signal path including an exemplary adaptive high pass post-filter and response in accordance with various exemplary and alternative exemplary embodiments;

FIG. 6 is a flow chart illustrating an exemplary speech quality test procedure in accordance with various exemplary and alternative exemplary embodiments;

FIG. 7 is a diagram illustrating portions of an exemplary test interface in accordance with various exemplary and alternative exemplary embodiments; and

FIG. 8 is a block diagram illustrating components of an exemplary apparatus in accordance with various exemplary and alternative exemplary embodiments.

### DETAILED DESCRIPTION OF THE PREFERRED EMBODIMENTS

In overview, the present disclosure concerns communications devices or units, often referred to as communication units, such as telephone handsets, cellular telephone or two-way radio handsets, portable music players such as MP3 players, portable computers, PDAs and the like having audio capability. More particularly, various inventive concepts and principles are embodied in communication devices and other audio-capable devices, and methods therein for self adjusting audio levels and equalization. It should be noted that in addition to connoting a typical handset or audio device such as a player, the term communication device or communication unit may be used interchangeably with subscriber unit, wireless subscriber unit, wireless subscriber device or the like. Each of these terms denotes a device ordinarily associated with a user and typically a wireless mobile device that may be used with a public network, for example in accordance with a service agreement, or within a private network such as an enterprise network. Examples of such units include personal digital assistants, personal assignment pads, and personal computers equipped for wireless operation, a cellular handset or device, or equivalents thereof provided such units are arranged and constructed for operation using audio.

The instant disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

It is further understood that the use of relational terms such as first and second, and the like, if any, are used solely to distinguish one from another entity, item, or action without necessarily requiring or implying any actual such relationship or order between such entities, items or actions.

Much of the inventive functionality and many of the inventive principles when implemented, are best supported with or in software or integrated circuits (ICs), such as a digital signal processor and software therefore or application specific ICs. It is expected that one of ordinary skill, notwithstanding possibly significant effort and many design choices motivated by, for example, available time, current technology, and economic considerations, when guided by the concepts and principles disclosed herein will be readily capable of generating such software instructions or ICs with minimal experimentation. Therefore, in the interest of brevity and minimization of any risk of obscuring the principles and concepts according to the present invention, further discussion of such software and ICs, if any, will be limited to the essentials with respect to the principles and concepts used by the preferred embodiments.

In addition to devices of a general nature with audio capability, the communication devices of particular interest are those providing or facilitating voice/audio communications services over cellular wide area networks (WANs), such as conventional two way systems and devices, various cellular phone systems including analog and digital cellular, CDMA (code division multiple access) and variants thereof, GSM, GPRS (General Packet Radio System), 2.5 G and 3 G systems such as UMTS (Universal Mobile Telecommunication Service) systems, Internet Protocol (IP) Wireless Wide Area Networks like 802.16, 802.20 or Flarion, integrated digital enhanced networks and variants or evolutions thereof. Furthermore the wireless communication units or devices of interest can have short range wireless communications capability normally referred to as WLAN capabilities, such as IEEE 802.11, Bluetooth, or Hiper-Lan and the like preferably using CDMA, frequency hopping, OFDM or TDMA access technologies and one or more of various networking protocols, such as TCP/IP (Transmission Control Protocol/Internet Protocol), UDP/UP (Universal Datagram Protocol/Universal Protocol), IPX/SPX (Inter-Packet Exchange/Sequential Packet Exchange), Net BIOS (Network Basic Input Output System) or other protocol structures. Alternatively the wireless communication units or devices of interest may be connected to a LAN using protocols such as TCP/IP, UDP/UP, IPX/SPX, or Net BIOS via a hardwired interface such as a cable and/or a connector.

As further discussed herein below, various inventive principles and combinations thereof are advantageously employed to provide self adjustment of audio levels and equalization.

In accordance with various exemplary embodiments, audiogram, loudness, or hearing profile tests, or the like, are conducted to determine a listener's hearing characteristics, such as a hearing loss profile. Since, as described above, a typical communication unit has a band-limited frequency response typically from around 150 Hz to around 3600 Hz, and may also have a standard audio equalization configuration which can alter the basic audio frequency response of the device, it is necessary to conduct tests on individual devices to allow response factors to be addressed and overcome on a per device basis. When a listener's hearing capabilities, impairments, preferences, or the like are determined, an audio profile is generated and will be used on the communication device for conditioning audio output thereafter. It will further be appreciated that depending on the output device which can be, for example, a speaker, an audio transducer such as a piezo-electric transducer, a headset speaker, earpiece, or the like, a user may wish to alter the default equalization for the particular audio output device being used.

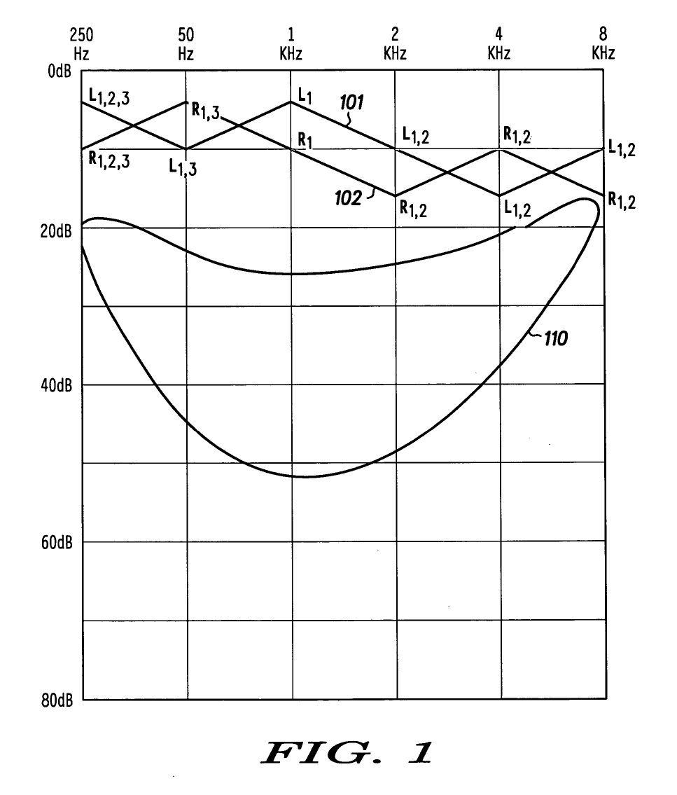

In order to generate an audio profile, all or significant ones of the frequencies which the communication device is capable of generating must be tested during hearing profile tests. Thus exemplary hearing profile tests use a standard tone sweep to generate a listener's audiogram as will be appreciated by those of skill in the art. Referring now to FIG. 1, a representative audio profile associated with a typical listener will be discussed and described. In an audiogram, a sweep of frequencies from 250 Hz to 8 kHz are typically tested at one octave intervals of 250 Hz, 500 Hz, 1 KHz, 2 KHz, 4 KHz and 8 KHz. Hearing levels at every octave are tested starting from a reference level which is clearly audible. Normal hearing levels range from 0 dB to 20 dB in all frequencies. In a conventional audiography screening session, 2 tones of 256 Hz and 4,096 Hz are delivered at 5 and 10 dB falling within the normal speech range. Detailed audiography shows that in listeners with normal hearing, low tones (around 64 Hz) are heard at 1 or 2 dB and high tones (11,584 Hz) are heard at around 10 dB, and tones in between around 64 Hz and 11,584 Hz are heard at less than 10 dB. For reference, a whisper is about 20 dB, loud music is around 80 to 120 dB, and a jet engine is about 140 to 180 dB. The level of test signal can then be attenuated toward, for example a 0 decibel (dB) reference level, until the signal is no longer perceived. As will be appreciated by one of ordinary skill in the art, a decibel is a logarithmic measure of relative sound energy or relative signal strength or attenuation from a reference level typically designated as 0 dB. Typical profiles can be generated for left ear response 101 and right ear response 102 as shown in FIG. 1 in connection with a normal response zone 110.

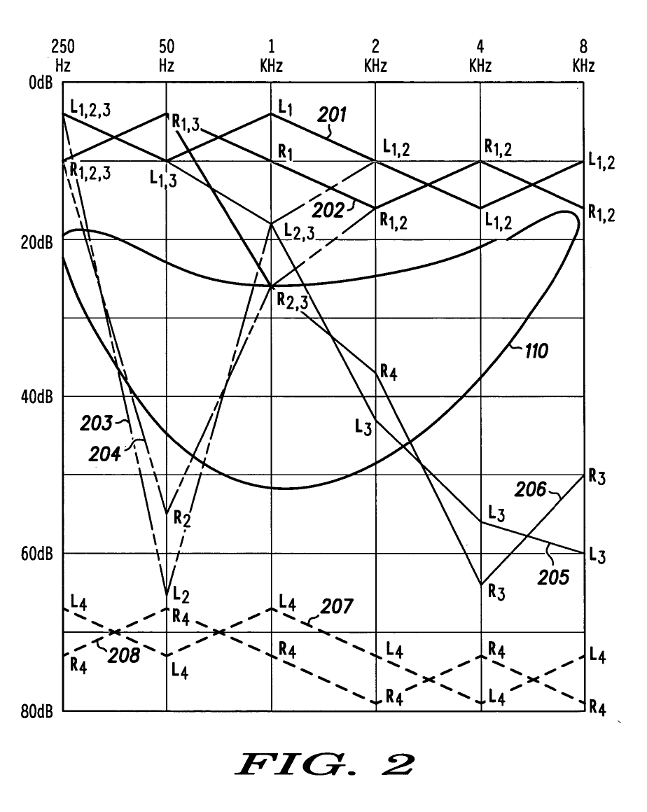

While left ear response 101 and right ear response 102 represent normal hearing profiles, various exemplary abnormal profiles are shown in FIG. 2. In addition to providing adjustments to “normal” hearing conditions, the present invention may be of particular use to the hearing impaired. Hearing loss affects approximately 16 million Americans with over 80% of hearing loss being sensorineural, meaning the loss results from damaged hair cells in the cochlea and cannot generally be improved by medical or surgical treatment. For example, a low frequency deficiency is shown as profile 2 or left ear response 203 and right ear response 204. Profile 3 shows a high frequency deficiency with left ear response 205 and right ear response 206. Profile 4 shows a total hearing deficiency across the frequency spectrum with left ear response 207 and right ear response 208.

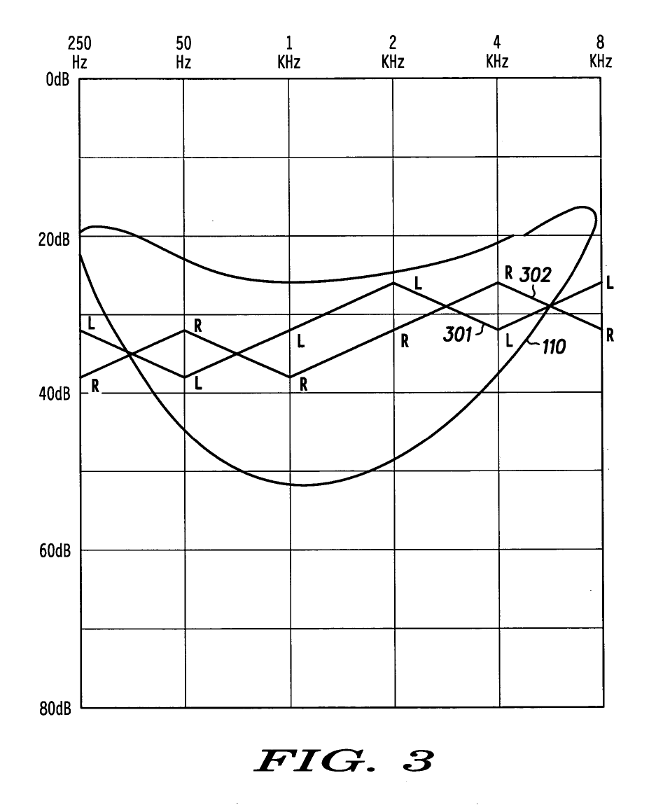

It should be noted that the objective of self adjusting in accordance with various exemplary embodiments is to bring the hearing levels to within normal zone 110 as shown in FIG. 3. Thus, while left ear response 301 and right ear response 302 are shown more or less within normal zone 110, the original responses could have included a deficiency as previously described in connection with, for example, FIG. 2.

Although the limits of human hearing are generally considered to be from around 20 Hz to around 20 KHz, testing in accordance with various exemplary embodiments need only support the available bandwidth of the communication unit or device which as noted is around 150 Hz to around 3,600 Hz for a typical wireless communication unit. Accordingly, a Graphical User Interface (GUI) is used to present the audiogram test to, for example, a user or listener. Since loudness is a function of level and frequency, the exemplary audiogram test will need to be conducted at each frequency for various volume levels associated with the communication unit or device. While the diagrams shown in the above figures have profiles for the left and right ear, an average unimpaired listener's profile is closely matched in both ears and thus the audiogram will generate a profile based on the hearing of whatever ear is used during the listening test. An impaired user will likely conduct the test with the ear having the least degree of impairment. The listener decisions to increase or lower volume will thus be conditioned by the audio profile generated from the audiogram level tests and information associated therewith will be used to condition each frequency.

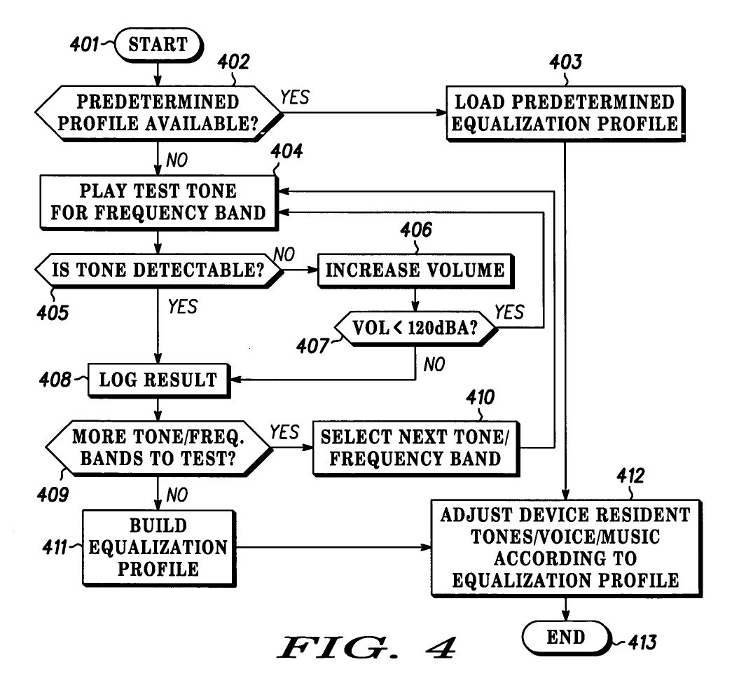

In an exemplary audiogram test procedure, for example, as illustrated in FIG. 4, after starting at 401, a determination may be made as to whether an equalization profile has already been generated or a predetermined equalization profile exists at 402. If the equalization profile exists, it can be loaded at 403, for example after a prompt provided in a Graphical User Interface (GUI) which is described in greater detail hereinafter, at which point the loaded equalization profile is used at 412 to self-adjust, adjust, or otherwise condition an audio signal output which may consist of music, tones, speech or the like. If however, no predetermined or generated profile is found to exist, a sequence of tones is presented to a listener, for example, one at a time from a low frequency tone to a high frequency tone for each volume level in the audiogram test at 404. If the tone is determined to be not detected at 405, level or volume is increased at 406 and a test is performed to determine whether the level is within the limits of 120 dB_{average} at 407. If so, the tone is replayed at 404 at the new level and the test for detection is repeated at 405 until the maximum level is reached or the tone is heard or detected. If the tone is determined to be detectable at 405, the result is logged at 408 and it is determined whether additional frequencies require testing at 409. If additional frequencies remain to be tested, the next tone is selected at 410 and replayed at 404 and so on. If no additional frequencies remain to be tested, then the equalization profile is built at 411 at which point the newly generated equalization profile is used at 412 to self-adjust, adjust, or otherwise condition an audio signal output which may consist of music, tones, speech or the like. The exemplary audiogram or loudness test may then be terminated at 413. It will be appreciated that after testing is completed the generated audio equalization profile can be used thereafter to adjust the audio output. However, the test may be run again to generate a new equalization profile by invoking the audiogram or loudness test from, for example, a GUI menu as will be described in greater detail hereinafter.

It should be noted that test tones can be spaced on a critical band scale since loudness is based on the critical band concept of hearing. A table of critical band frequencies for the entire range of typical human hearing is given in table 1 below. One of ordinary skill in the art will appreciate that while table 1 shows all the critical band frequencies, not all the critical band frequencies will be relevant in all devices. For example, while a relatively high fidelity audio player may have a frequency response across the entire range of hearing, a typical communication unit will have a band limited frequency response.

TABLE 1
Critical band frequency scale

| | | | center |
|---|---|---|---|
| Critical | frequency | bandwidth | freq |
| band # | (Hz) | (Hz) | (Hz) |
| 1 | 100 | 100 | 50 |
| 2 | 200 | 100 | 150 |
| 3 | 300 | 100 | 250 |
| 4 | 400 | 100 | 350 |
| 5 | 510 | 110 | 450 |
| 6 | 630 | 120 | 570 |
| 7 | 770 | 140 | 700 |
| 8 | 920 | 150 | 840 |
| 9 | 1080 | 160 | 1000 |
| 10 | 1270 | 190 | 1170 |
| 11 | 1480 | 210 | 1370 |
| 12 | 1720 | 240 | 1600 |
| 13 | 2000 | 280 | 1850 |
| 14 | 2320 | 320 | 2150 |
| 15 | 2700 | 380 | 2500 |
| 16 | 3150 | 450 | 2900 |
| 17 | 3700 | 550 | 3400 |
| 18 | 4400 | 700 | 4000 |
| 19 | 5300 | 900 | 4800 |
| 20 | 6400 | 1100 | 5800 |
| 21 | 7700 | 1300 | 7000 |
| 22 | 9500 | 1800 | 8500 |
| 23 | 12000 | 2500 | 10500 |
| 24 | 15500 | 3500 | 13500 |

In accordance with various exemplary embodiments, the exemplary GUI can provide a representation of each tone tested for and display it to the screen so the listener can see the results. The communication unit further can keep, for example, a history of audiograms for display and comparison. In accordance with other exemplary embodiments, the GUI can display a chart of the frequency response profile showing the hearing loss attenuation over each critical band.

As noted, the audiogram test will produce a profile containing all values and perceived attenuation levels across the frequency band for the particular communication unit. Since each individual communication unit may have its own unique frequency response profile, based for example on differences between component tolerances and the like, it is necessary to do listening tests on the actual communication unit. The listener's tonal sensitivity curve, as determined by the listening test and the resulting audiogram and profile, determines the required level scaling for the critical band tones and can be used to determine an equalization profile which compensates or otherwise restores the individual's hearing to or near to a normal hearing profile. The equalization profile can be used in all subsequent audio processing and signal output functions. The equalization profile, as will be appreciated by one of ordinary skill in the art, specifies how much attenuation or amplification is necessary to balance the loudness of frequency components across the band for the communication unit. Since tones are used, the above described audiogram or listening test provides a relatively coarse adjustment of audio levels. A finer adjustment may further be desirable for making characteristics associated with speech more discemable.

In addition, the audiogram can be used to address compression by determining “headroom” which can be defined as the degree of amplification possible for a frequency before saturation occurs. As will be understood by one of ordinary skill in the art, saturation occurs when gain levels are sufficiently high to cause an amplifier to operate outside its linear region resulting in non-linearity and clipping in the audio output signal. For a typical device, a one-to-one correspondence between the input and output levels should be present. At input levels above some level, for example 10 dB, compression effects can occur.

Voice conditions can be categorized according to parameters including formants, fricatives, and equalization. Formants are resonant sounds having distinguishing frequency components allowing, for example, vowel sounds to be distinguished from each other. Fricatives are sounds produced by air flowing through a narrow channel made by two articulating organs in close proximity such as the tip of the tongue and the upper teeth. The turbulent airflow resulting from the narrow passage produces a characteristic noise called “frication”. Equalization is the relative emphasis across the frequency spectrum. Ideally, equalization will emphasize weak frequencies and de-emphasize strong frequencies and should result in a “flat” response in the listener where all frequencies are heard with equal emphasis. Due to impairments, preferences or the like, certain frequencies will require greater emphasis to achieve the desired response in the listener.

In accordance with various exemplary embodiments, voice conditions noted above, while associated with frequencies generated in the testing described hereinabove, may produce anomalies which can affect intelligibility and which can be corrected for in an additional fine adjustment test involving, for example, speech oriented listening and intelligibility tests rather than tone-based audiogram or loudness tests. Thus, in an exemplary test for nasalty and formant sharpening, nasal sounds can be presented and a formant postfilter adjusted until, for example, improved recognition is attained in intelligibility. In an exemplary consonant and midfrequency emphasis test, certain fricitaves are presented and tested for intelligibility. The midfrequency amplitudes for example can be sweeped in level until recognition results improve. In an exemplary unvoiced speech and audio equalization test the entire frequency band is tested for loudness or level imbalances. For example, when high frequencies are overemphasized, ‘s’ sounds can be harsh and piercing. Accordingly, the audiogram or loudness tests described above may be conducted to establish acceptable volume levels and additional adjustments can be made to soften harsh consonants and fricatives, and re-shape formants.

In addition to hearing difficulties, speech disorders including nasality may affect the quality of the speech being generated and input to a communication unit; where this speech is ultimately destined for transmission to a terminating communication unit and thus listened to by others. Speech studies of people with nasal speech disorders reveal that nasality is primarily due to pronounced abnormal resonances in the nasal cavity which amplify formant energy. Other nasal related disorders include the loss of consonant articulation due to an inability to build air pressure because of air escaping through the nasal cavity. It is envisioned that principals discussed and described herein could be used to compensate for nasality during processing of the speech signal in, for example, an exemplary vocoder or the like.

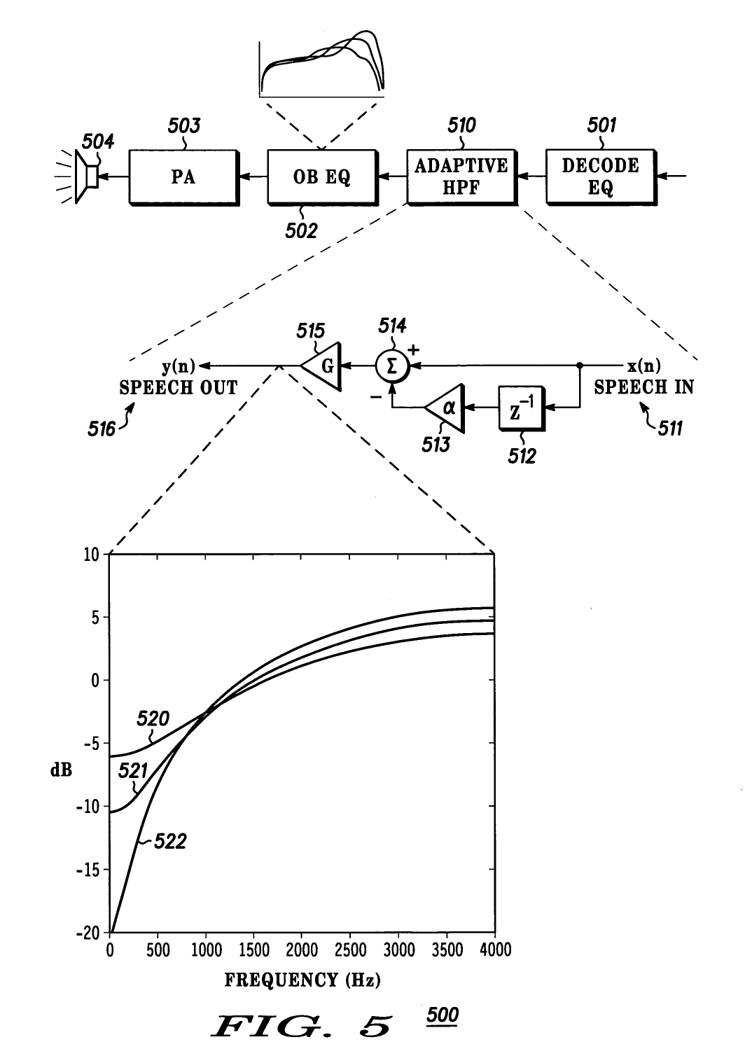

As can be seen in exemplary block diagram 500 of FIG. 5, a communication unit in accordance with various exemplary embodiments, can be equipped with adaptive postfilter 510 for adjusting spectral tilt, adjusting the degree of voicing through formant sharpening, or simply changing the audio equalization profile. A speech signal is decoded in decoder 501 and samples are input to adaptive postfilter 510. The output of postfilter 510 is passed through equalizer 502 configured to adjust the audio signal according to an equalization profile which can be loaded or generated and applied in the manner described above. The output of equalizer 502 is passed through power amplifier 503 and output through audio transducer 504 which can be a speaker or the like. Postfilter 510 can be constructed of standard filter elements which operate on speech signal x(n) 511 which may be a series of digital speech samples as may be decoded from an exemplary vocoder or the like, and as noted may already be conditioned by an equalization profile which can be associated with decoder 501. Samples associated with speech signal x(n) 511 may be input to filter element 512 which may be a digital filter element as is known in the art. A filter parameter α may be derived based on the desired compensation associated with formant sharpening, spectral tilt or the like and the parameters of the filter in block 513 whereupon the input speech signal x(n) 511 may be summed at summer 514 with the output samples from filter element 512 and parameter block 513. Finally a gain element 515 may add a suitable level adjustment to the signal samples prior to being output as output speech signal y(n) 516 to, for example, equalizer 502. As will be appreciated by one of ordinary skill in the art, depending on values associated with α, the output speech signal y(n) 516 will be generated according to exemplary response 520 for α=0.5, exemplary response 521 for α=0.7, and exemplary response 522 for α=0.9.

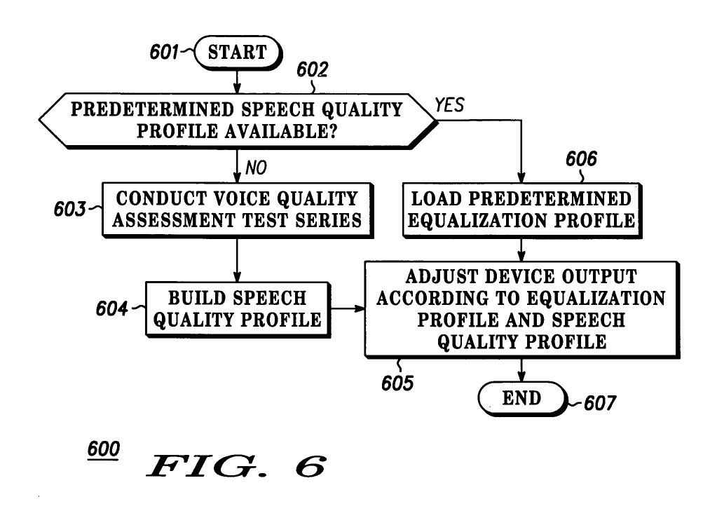

It should be noted that while some postfiltering may be present and associated with default profiles, postfilters are not typically accessible for reconfiguration or adjustment such as in accordance with the present invention. Thus, a more accessible environment is needed where a listener can perform specific listening tests during an exemplary test period to provide an even finer adjustment over the tone sweeping audiogram tests already described. An exemplary procedure 600 associated with performing speech quality assessment tests such as may be presented to a user by way of a GUI is shown and described in connection with FIG. 6. After starting at 601, it can be determined whether a predetermined speech quality profile is available, such as a speech quality profile generated from a previous test or the like in accordance with various exemplary embodiments. If the speech quality profile is available, it can be loaded at 606 and used, for example, in connection with the equalization profile described herein above to adjust the audio output of the device particularly for speech. If no previously generated speech quality profile is available, a series of tests may be conducted at 603. Each test result reveals a certain attribute of voice quality which can be associated with a frequency or weighting profile. For instance, the intelligibility of certain fricative sounds predominant in the 2-3 KHz range can be improved by a slight accentuation of the 2-KHz range. Such accentuation could be applied during a fine adjustment intelligibility test to observe whether the recognition response is improved and if so, the speech quality profile can be generated at 604 and applied or otherwise included at 605 in audio processing before or after the equalization profile, or the like. If, in accordance with various exemplary embodiments, the present invention is embodied in, for example, a digital signal processor, it may be that the postfilter and equalization profiles can be inter-related and accomplished using common elements or by making specific adjustments to the coarse adjustment described above without adversely affecting the overall equalization. Exemplary audiogram or loudness tests could be used in connection with tests associated with finer adjustments to determine the optimum amount of attenuation using, for example, a d'Esser filter. For example intelligibility tests when conducted can determine whether certain vowel sounds are too nasal sounding, hence formant sharpening could be reduced and the like.

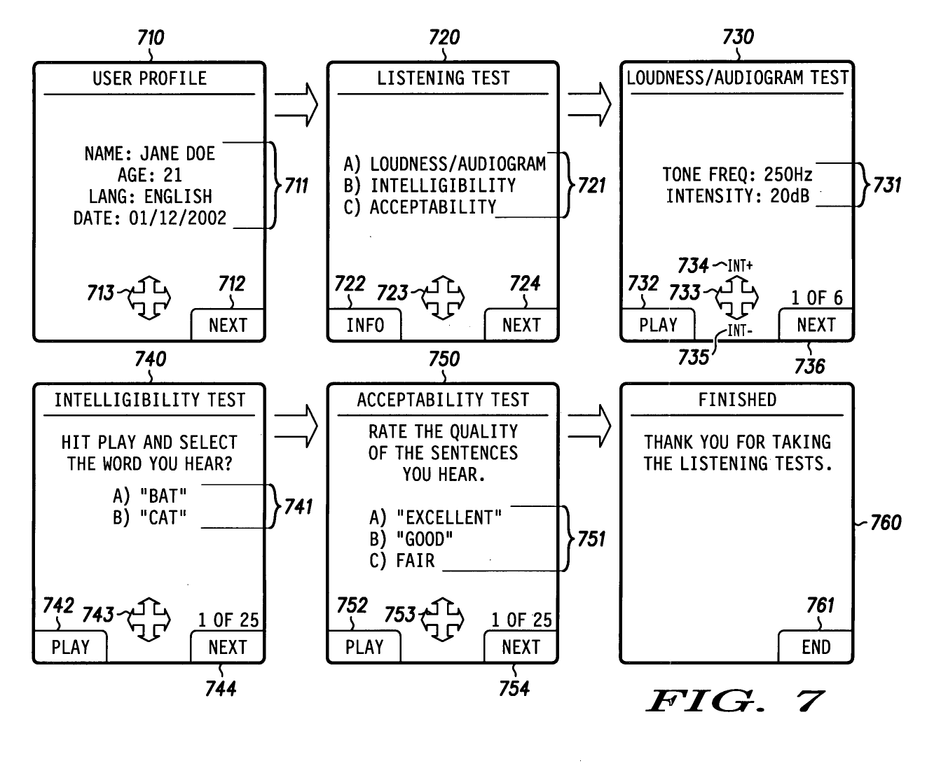

Since conventional phones or communication devices do not typically employ tests to automatically set a user profile to achieve individually tailored responses, performance, loudness, intelligibility, and acceptability tests can be conducted on an exemplary communication unit in accordance with various exemplary embodiments, using an interactive test procedure which can be presented to a user through a user interface such as a test suite presented using Graphical User Interface (GUI) 700 as shown in FIG. 7 and discussed and described hereinafter. Tests 710-760 allow for adjustment of the audio equalization or spectral tilt settings using parameters attained during the listening tests and provide, for example in test 730 the loudness or audiogram test described herein above. In essence, listening tests are performed on the phone and the phone automatically adjusts the frequency response using the speech quality profile as it receives user feedback during tests 710-760.

In accordance with an alternative exemplary embodiment (not shown), tests may be performed, for example during manufacturing, using external equipment such as an external listening test suite to evaluate speech processed by the combined enhancement algorithms. Accordingly, a serial port interface can be used to stream speech data from and to the exemplary communication unit with an accessory cable in order to present an adequate opportunity to perform adjustments. Software to uncompress/compress and encode/decode streamed speech exists on the communication unit for performing processing. A recording test can be used to record speech utterances to be processed for listening tests. Vocoded speech data may be acquired from the communication unit and may be processed externally with, for example, loudness enhancement algorithms. The processed and vocoded speech can be uploaded back to the communication unit and can be used to conduct the listening tests as noted.

With continuing reference to GUI 700 shown in FIG. 7, test blocks 710-760 are now described in greater detail. It will be appreciated that loudness or audiogram test 730, intelligibility test 740, and acceptability test 750 can be combined into a suite of tests available via the GUI of the communication unit and can be referred to, for example, as listening test 720. Each listening test can have its own display for presenting data and receiving input for controlling and advancing the test procedure. In test block 710, a user profile screen may be presented so a listener can enter name, age, native language and other information 711 which may be used to store profile information or, in some exemplary embodiments to provide additional information to improve the quality of the test and ultimately of the generated profile information. For example, in accordance with various alternative exemplary embodiments, if the data from test block 710 indicates that the test subject is a female, additional known characteristics associated with the female voice can be used to better condition, for example, an output signal generated from the device or communication unit. In addition, information such as the date of the test can be automatically recorded and used to tag the results. After information 711 is entered, next button 712 may be pressed to advance to the next block.

As noted, the listening test block 720 enumerates the type of tests available. In accordance with various exemplary embodiments, three listening tests are shown in information area 721: loudness or audiogram test 730, intelligibility test 740 and acceptability test 750. The listener ideally will take all three tests although the user may return to a single test if desired. “Info” button 722 may be used to present the rules for each test, while “Arrow” button 723 can be used to select between tests, and once a test is selected, “Next” button 724 can be used to begin the selected test. It will be appreciated that the user can at any time return to listening test block 720 if the fidelity of the device or communication unit requires improvement.

In loudness or audiogram test 730, a tone at a particular level, both the tone frequency and level being displayed in information area 731, may be presented to a listener to judge for loudness. The listener can increase or decrease loudness for the tone using arrow key 733 in the “Int+” direction 734 or the “Int−” direction 735. When the loudness is determined to be sufficient for the listener “Next” button 736 can be pressed which tabulates the intensity level for the particular tone and the next tone can be tested. When the final tone is tested and logged, pressing “Next” button 736 will exit loudness or audiogram test 730.

In intelligibility test 740, the listener can be presented with 25 words in information area 741 to judge for intelligibility. The user presses “Play” button 742 and the word is played. After playing, two words can be displayed in information area 741, at which point arrow button 743 can be used to select which word was heard. It will be appreciated that the word choices are not presented in text form until the word is played since the listener must decide what word was said. The listener is further not given information as to which word was processed, and the presentation order of the words after playing is random. It should be noted that the intelligibility test 740 can add artificial noise to the word in an effort to generate possible ambiguities between for example consonants and fricatives or the like, as might exist between the words “fat” and “sat”. Results are tabulated and the user presses “Next” button 744 to proceed to the next word. When no more words are available, “Next” button 744 may be used to advance to the next test.

In acceptability test 750, the listener is asked to provide a quality rating of the speech they hear in exemplary sentences. In accordance with various exemplary embodiments, 20 sentences are provided to evaluate acceptability. A sentence is played by pressing “Play” button 752 and the user is asked to rate the quality of the speech they hear in accordance with ratings such as “excellent”, “good”, “fair”, or the like as may be presented in information area 751, by moving to the displayed rating using arrow button 753. The sentences are selected and processed with the loudness enhancement algorithm at random. The listener rates the quality as “excellent”, “good”, or “fair”. Results are tabulated and the user proceeds to the next sentence for evaluation by hitting “Next” button 754. When no more sentences are available, “Next” button 754 will advance to “Finished” block 760 at which point the test suite may be exited by pressing “End” button 761.

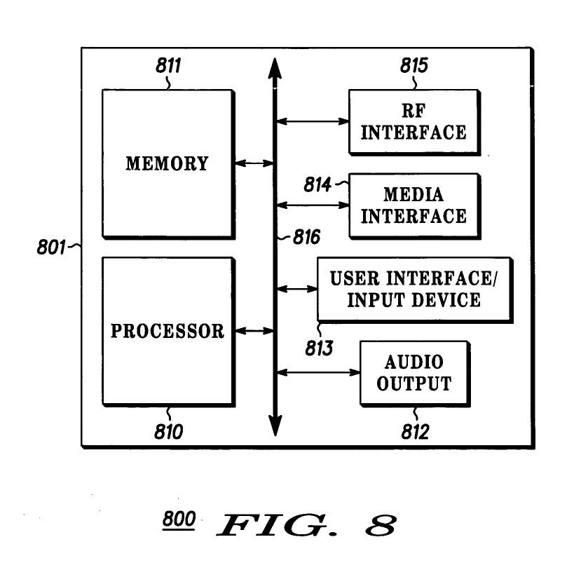

It will be appreciated that in accordance with various exemplary embodiments, the present invention may be implemented as an apparatus. Exemplary apparatus 800 shown in FIG. 8, will now be discussed and described. Device 801 which may be a communication unit or the like as described above, may include processor 810 and memory 811 coupled using bus 816. Processor 810 may be a general purpose processor or a dedicated processor such as a signal processor and may be implemented as a dedicated ASIC or the like as noted herein above. Memory 811 will be a memory device such as a Random Access Memory (RAM) which matches the transfer speed and access speed requirements associated with processor 810 and bus 816. In addition, device 801 can include RF interface 815, particularly where device 801 is a communication unit such as a wireless handset or the like. Media interface 814 can be an interface to, for example, a Compact Disc, or digital audio file storage device or the like for transferring music or other recorded audio, or alternatively may be an interface to a network such as the Internet for transferring recorded audio or even live audio. In order to display information and receive inputs, such as in the context of the GUI and test suites presented thereon as described above, device 801 also includes user interface 813 including at least one input device and a display. Audio output 812 may be used to output the audio signal during normal operation and during the tests described herein above. Audio output 812 includes output to a speaker, audio transducer, piezo-electric transducer, or the like, or may output an audio signal to a headphone or other remote listening device.

This disclosure is intended to explain how to fashion and use various embodiments in accordance with the invention rather than to limit the true, intended, and fair scope and spirit thereof. The invention is defined solely by the appended claims, as they may be amended during the pendency of this application for patent. The foregoing description is not intended to be exhaustive or to limit the invention to the precise form disclosed. The embodiment(s) was chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims, as may be amended during the pendency of this application for patent, and all equivalents thereof, when interpreted in accordance with the breadth to which they are fairly, legally, and equitably entitled.

What is claimed is:

1. A method for adjusting parameters associated with an audio signal output from a device, the method comprising:
outputting the audio signal from the device in accordance with a first interactive test profile presented on a user interface associated with the device during a first test period in which a first interaction with the user interface is capable of taking place to form a first adjustment profile having first adjustment levels associated with the parameters; and
outputting the audio signal from the device after the first test period such that the parameters are adjusted in accordance with the first adjustment levels of the first adjustment profile.

2. A method in accordance with claim 1, wherein the first interactive test profile includes a tone output at an adjustable level, and wherein the first interaction includes adjusting the adjustable level in accordance with a preference associated with a listener to form the first adjustment levels.

3. A method in accordance with claim 1, wherein the first interactive test profile includes loading a predetermined adjustment profile associated with a hearing impairment characteristic, the hearing impairment characteristic including one of a low frequency impairment, and a high frequency impairment, the predetermined adjustment profile having predetermined adjustment levels associated with the parameters, wherein the outputting the audio signal from the device after the first test period includes outputting the audio signal in accordance with the predetermined adjustment levels of the predetermined adjustment profile.

4. A method in accordance with claim 1, wherein the first interactive test profile includes loading a predetermined adjustment profile associated with a user, the predetermined adjustment profile having predetermined adjustment levels associated with the parameters, wherein the outputting the audio signal from the device after the first test period includes outputting the audio signal in accordance with the predetermined adjustment levels of the predetermined adjustment profile.

5. A method in accordance with claim 1, wherein the parameters include an output level for each of a plurality of frequency bands, and wherein the outputting the audio signal from the device in accordance with a first interactive test profile during a first test period further includes:
outputting a test tone at the output level in the each of the plurality of frequency bands;
determining whether the test tone is acceptable at the output level by detecting a first interaction with the device;
adjusting the output level if the test tone is not acceptable and outputting the test tone at the output level according to the adjusting; and
determining whether the test tone is acceptable at the output level according to the adjusting by detecting a second interaction with the device.

6. A method in accordance with claim 5, wherein the adjusting the output level includes limiting the increasing the output level such that the audio signal output from the device does not exceed a predetermined level, the predetermined level including a 120 decibel (dB) average level.

7. A method in accordance with claim 5, wherein the test tone includes a warbled test tone.

8. A method in accordance with claim 1, further comprising:
outputting the audio signal from the device in accordance with the first adjustment profile and a second interactive test profile presented on a user interface associated with the device during a second test period in which an interaction with the user interface is capable of taking place to form a second adjustment profile having second adjustment levels associated with the parameters; and
outputting the audio signal from the device after the second test period such that the parameters are adjusted in accordance with the second adjustment levels of the second adjustment profile.

9. A method in accordance with claim 5, wherein the plurality of frequency bands includes frequency bands of approximately 250 Hz, 500 Hz, 1000 Hz, 2000 Hz, 4000 Hz, and 8000 Hz.

10. A method in accordance with claim 1, wherein the device includes a communication unit.

11. A method in accordance with claim 1, wherein the device includes one of: a wireless communication handset, a wired communication handset, an MPEG 1 (Motion Picture Experts Group) Layer 3 (MP3) player, a Compact Disc (CD) player, and a digital audio player.

12. An apparatus for adjusting parameters associated with an audio signal in a device, the apparatus comprising:
an audio output device;
an input device;
a memory storing a first interactive test profile; and
a processor coupled to the audio output device, the input device, and the memory, the processor configured to facilitate:
retrieving the first interactive test profile from the memory, the first interactive test profile including a test tone for each of a plurality of frequency bands and an audio level associated with the test tone;
outputting the test tone for the each of the plurality of frequency bands at the audio level using the audio output device;
determining based on a first input from the input device whether the test tone was detectable at the audio level; and
adjusting the audio level if the determining based on the first input from the input device determines the test tone was not detectable,
wherein the outputting, the determining, and the adjusting occur during a first test period, and the processor, in the outputting, the determining, and the adjusting is further configured to form a first adjustment profile having first adjustment levels associated with the parameters based on the outputting, the determining, and the adjusting.

13. An apparatus in accordance with claim 12, wherein the processor is further configured to output the audio signal using the audio output device after the first test period such that the parameters are adjusted in accordance with the first adjustment levels of the first adjustment profile.

14. An apparatus in accordance with claim 12, wherein processor is further configured to facilitate:
retrieving a second interactive test profile from the memory, the second interactive test profile including a speech sample and an intelligibility parameter associated with the speech sample;
outputting the speech sample using the audio output device;
determining based on a second input from the input device whether the speech sample was intelligible according to the intelligibility parameter; and
adjustinging the intelligibility parameter if the determining based on the input from the input device determines the speech sample was not intelligible,
wherein the outputting, the determining, and the adjusting occur during a second test period, and the processor, in the outputting, the determining, and the adjusting is further configured to form a second adjustment profile having second adjustment levels associated with the parameters based on the outputting, the determining, and the adjusting.

15. An apparatus in accordance with claim 14, wherein the intelligibility parameter includes one of a spectral tilt for an entire audio spectrum associated with the device and a formant sharpening profile.

16. An apparatus in accordance with claim 12, wherein the device includes a one of: a wireless communication unit, a wired communication handset, an MPEG 1 (Motion Picture Experts Group) Layer 3 (MP3) player, a Compact Disc (CD) player, and a digital audio player.

17. An apparatus in accordance with claim 12, wherein the processor, in adjusting the audio level includes limiting the increasing the audio level such that the audio signal output from the device does not exceed a predetermined level, the predetermined level including a 120 decibel (dB) average level.

18. A method for adjusting an audio signal output from a communication unit, the method comprising:
outputting the audio signal from the communication unit in accordance with a test profile during a first test period in which interaction is capable of taking place to form an adjustment profile having adjustment levels associated with a plurality of frequency bands of the audio signal; and
outputting the audio signal from the communication unit after the first test period such that the audio signal is adjusted in accordance with the adjustment levels of the adjustment profile.

19. A method in accordance with claim 18, further comprising loading a predetermined adjustment profile associated with a hearing impairment characteristic, the hearing impairment characteristic including one of a low frequency impairment, and a high frequency impairment, the predetermined adjustment profile having predetermined adjustment levels associated with the parameters, wherein the outputting the audio signal from the communication unit after the first test period includes outputting the audio signal in accordance with the predetermined adjustment levels of the predetermined adjustment profile.

20. A method in accordance with claim 18, further comprising loading a predetermined adjustment profile associated with a user, the predetermined adjustment profile having predetermined adjustment levels associated with the parameters, wherein the outputting the audio signal from the communication unit after the first test period includes outputting the audio signal in accordance with the predetermined adjustment levels of the predetermined adjustment profile.
